(19) United States
(12) Patent Application Publication
(10) Pub. No.: US 2026/0066075 A1
(43) Pub. Date: Mar. 5, 2026

(54) INDOOR SPACE PROVISION SYSTEM AND INDOOR SPACE PROVISION METHOD

(71) Applicant: Hitachi, Ltd., Tokyo (JP)

(72) Inventors: Hang LIU, Tokyo (JP); Shintaro SUZUKI, Tokyo (JP); Akihiko HYODO, Tokyo (JP)

(21) Appl. No.: 19/211,929

(22) Filed: May 19, 2025

(30) Foreign Application Priority Data
Aug. 29, 2024 (JP) 2024-147565

(51) Int. Cl. G16H 20/00; G06F 40/40; G06V 20/52; G06V 40/20

(52) U.S. Cl. CPC G16H 20/00; G06F 40/40; G06V 20/52; G06V 40/20

(57) ABSTRACT

An indoor space provision system includes an indoor space monitoring unit configured to monitor a state of an indoor space, an indoor space state analysis unit configured to analyze the state of the indoor space and acquire indoor space state information as an analysis result, a user monitoring unit configured to monitor a mental/physical state of a user in the indoor space, a user state analysis unit configured to analyze the mental/physical state of the user and acquire user state information as an analysis result, and a recommendation tag creation unit configured to create, based on the indoor space state information and the user state information, a recommendation tag related to an indoor space that affects a behavior of the user.

### CROSS-REFERENCE TO RELATED APPLICATION

The present application claims priority from Japanese application JP2024-147565, filed on Aug. 29, 2024, the content of which is hereby incorporated by reference into this application.

### BACKGROUND OF THE INVENTION

### 1. Field of the Invention

The present invention is suitably applied to an indoor space provision system and an indoor space provision method related to a technique for providing an indoor space comfortable for a user.

### 2. Description of Related Art

As a method for recommending activities based on mental health of a user, there is a technique disclosed in PTL 1. The technique disclosed in PTL 1 grasps an emotional state of a user based on a facial expression acquired by facial expression recognition, and recommends an activity together with a content to the user in order to improve the emotional state of the user.

### CITATION LIST

### Patent Literature

PTL 1: US2023/0317246

### SUMMARY OF THE INVENTION

However, in the technique described in PTL 1, although the emotional state of the user is improved by recommending the activity together with the content to the user, it is not possible to recommend an indoor space suitable for the mental/physical state of the user.

The invention has been made in view of the above points, and is intended to propose an indoor space provision system and an indoor space provision method capable of recommending an indoor space suitable for a mental/physical state of a user.

In order to solve such a problem, an indoor space provision system according to the invention includes: an indoor space monitoring unit configured to monitor a state of an indoor space; an indoor space state analysis unit configured to analyze the state of the indoor space and acquire indoor space state information as an analysis result; a user monitoring unit configured to monitor a mental/physical state of a user in the indoor space; a user state analysis unit configured to analyze the mental/physical state of the user and acquire user state information as an analysis result; and a recommendation tag creation unit configured to create, based on the indoor space state information and the user state information, a recommendation tag related to an indoor space that affects a behavior of the user.

In addition, an indoor space provision method according to the invention includes: an indoor space monitoring step of monitoring a state of an indoor space by an indoor space monitoring unit; an indoor space state analysis step of analyzing the state of the indoor space and acquiring indoor space state information as an analysis result by an indoor space state analysis unit; a user monitoring step of monitoring a mental/physical state of a user in the indoor space by a user monitoring unit; a user state analysis step of analyzing the mental/physical state of the user and acquiring user state information as an analysis result by a user state analysis unit; and a recommendation tag creation step of creating, based on the indoor space state information and the user state information, a recommendation tag related to the indoor space that affects a behavior of the user by a recommendation tag creation unit.

The invention can recommend an indoor space suitable for a mental/physical state of a user.

### BRIEF DESCRIPTION OF THE DRAWINGS

FIG. 1 is a system configuration diagram illustrating a configuration example of a smart building system as an example of an indoor space provision system according to a first embodiment;

FIG. 2 is a flowchart illustrating an example of a procedure of an indoor space provision process as an indoor space provision method in the smart building system;

FIG. 3 is a diagram illustrating an example of input and output on a user analysis side in the smart building system; and

FIG. 4 is a diagram illustrating an example of input and output on the user analysis side in the smart building system.

### DESCRIPTION OF EMBODIMENTS

Hereinafter, an embodiment of the invention will be described in detail with reference to the drawings.

### (1) First Embodiment

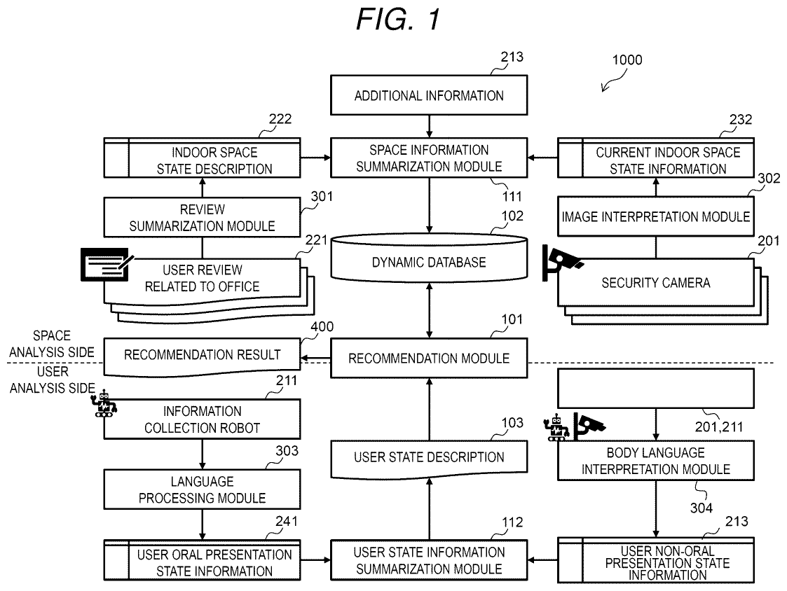

FIG. 1 is a system configuration diagram illustrating a configuration example of a smart building system 1000 as an example of an indoor space provision system according to a first embodiment. The smart building system 1000 is a system provided in a so-called smart building. The smart building is highly systematized, and has, for example, an office in which at least one indoor space is formed. In the present embodiment, the smart building includes, for example, a plurality of offices.

The smart building system 1000 includes, for example, a review summarization module 301, a space information summarization module 111, a security camera 201, an image interpretation module 302, a dynamic database 102, an information collection robot 211, a language processing module 303, a body language interpretation module 304, and a user state information summarization module 112, as a functional configuration thereof.

The smart building system 1000 uses, for example, a user review 221, an indoor space state description 222, and current indoor space state information 232 as data or the like. The user review 221 is a review described by the user and related to an indoor space of an office.

The user review 221 is input by the user using a user review input device (not illustrated). The indoor space state description 222 is a description related to a state of an indoor space of an office. The current indoor space state information 232 is information related to an indoor space of an office at a current time point.

The smart building system 1000 includes, for example, a user review input device, the security camera 201, and the information collection robot 211 as an indoor space monitoring unit that monitors a state of an indoor space of each office. Instead, the indoor space monitoring unit may be, for example, an indoor sensor (not illustrated) placed at any position in an indoor space, or an automatic cleaner (not illustrated) that automatically cleans an indoor space by detecting garbage on a floor with a sensor while autonomously traveling on the floor of the indoor space.

The user review input device is an input device for inputting a review of a user. The security camera 201 is a camera for visually capturing the user and the surrounding people including the user and their state.

The information collection robot 211 has a function of collecting surrounding voices and recognizing voices from voice data of the collected voice. Further, the information collection robot 211 is an interactive robot that interprets the content of a speech based on the recognized voice using artificial intelligence and outputs a response for the content of the speech in voice.

The smart building system 1000 includes, as a space analysis side, an indoor space state analysis unit that analyzes a state of an indoor space and acquires indoor space state information as an analysis result. The indoor space state information includes the indoor space state description 222 and the current indoor space state information 232. The indoor space state analysis unit includes the review summarization module 301 and the image interpretation module 302.

The review summarization module 301 summarizes the user review 221 related to, for example, an indoor space of an office A among the plurality of offices to acquire the indoor space state description 222 as the indoor space state information. The user review 221 related to the indoor space of the office A is a user review that is described by a user who has used the office A and is input using a user review input device.

The image interpretation module 302 acquires the current indoor space state information 232 as indoor space state information related to an indoor space monitored by the indoor space monitoring unit.

The smart building system 1000 includes a user state analysis unit as a user analysis side. The user state analysis unit analyzes a mental/physical state of the user and acquires user state information as an analysis result. The user state information includes user oral presentation state information 241 and user non-oral presentation state information 213. The user state analysis unit includes the language processing module 303 and the body language interpretation module 304.

The language processing module 303 executes language processing of the conversation uttered by the user and creates the user oral presentation state information 241 that is information related to a state of the user according to the oral presentation content of the user.

The body language interpretation module 304 interprets body language of the user and creates the user non-oral presentation state information 213 that is information related to a state of the user according to the non-oral presentation content of the user. Here, the body language refers to, for example, a content other than oral presentation (referred to as “non-oral presentation”) such as a gesture or behavior of the user, which indicates an intention, emotion, or mood of the user that can be recognized from the body or behavior of the user.

The user state analysis unit described above includes the user state information summarization module 112. The user state information summarization module 112 creates, based on the user oral presentation state information 241 and the user non-oral presentation state information 213, a user state description 103 related to a mental/physical state of the user.

The smart building system 1000 includes a recommendation module 101 as an example of a recommendation unit. When a specific recommendation tag created by the space information summarization module 111 as a recommendation tag creation unit matches a previous recommendation tag created in the past, the recommendation module 101 provides the user with the specific recommendation tag in consideration of the description content of the user state description 103. For example, the recommendation module 101 attaches the specific recommendation tag to a recommendation result 400 and provides the user with the recommendation result 400.

The language processing module 303 and the body language interpretation module 304 as an example of the user state analysis unit estimate a mental/physical state of the user based on a result of processing the content of a conversation of the user as a monitoring result by a user state monitoring unit using natural language processing. The user state analysis unit may use artificial intelligence instead of the natural language processing.

The configuration of the smart building system 1000 according to the present embodiment is as described above. Next, an indoor space provision method in the smart building system 1000 will be described.

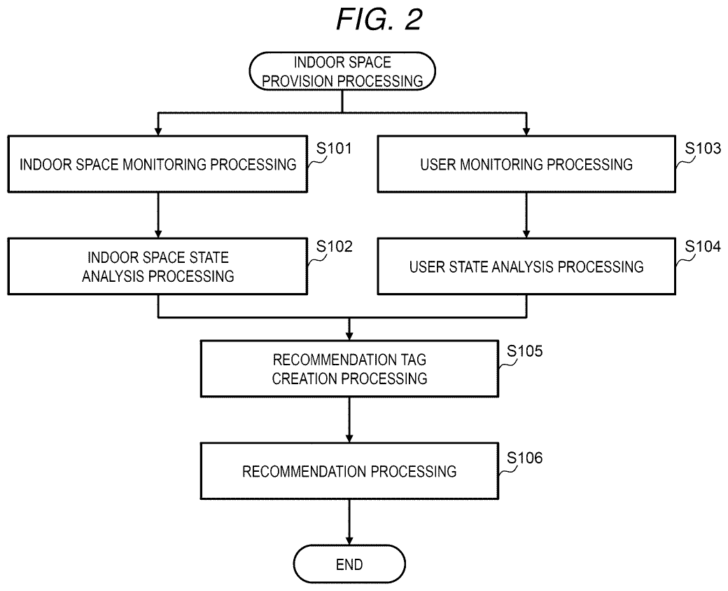

FIG. 2 is a flowchart illustrating an example of a schematic procedure of indoor space provision processing as an indoor space provision method in the smart building system 1000. Note that the following steps S101 and S102 may be executed before or simultaneously with steps S103 and S104.

### Indoor Space Monitoring Step

First, in step S101, the user review input device, the security camera 201, and the information collection robot 211 as an example of an indoor space monitoring unit monitor a state of an indoor space.

### Indoor Space State Analysis Step

In step S102, the review summarization module 301 and the image interpretation module 302 as an example of the indoor space state analysis unit analyze the state of the indoor space and acquire the indoor space state description 222 and the current indoor space state information 232 as an example of the indoor space state information as an analysis result.

In step S103, the security camera 201 and the information collection robot 211 as an example of a user monitoring unit monitor the state of the user by capturing an image of, for example, a conversation, a gesture, a facial expression, and a behavior as the mental/physical state of the user in the indoor space and performing recognition processing.

In step S104, the language processing module 303 and the body language interpretation module 304 as an example of a user state analysis unit analyze the mental/physical state of the user and acquire the user oral presentation state information 241 and the user non-oral presentation state information 213 as an example of user state information as an analysis result.

### Recommendation Tag Creation Step

In step S105, the space information summarization module 111 as an example of the recommendation tag creation unit creates, based on the indoor space state description 222 and the current indoor space state information 232 as an example of the indoor space state information, the user oral presentation state information 241 and the user non-oral presentation state information 213 as an example of the user state information, a recommendation tag related to the indoor space that may affect the behavior of the user.

Here, processing on the space analysis side of the smart building system 1000 will be described. On the space analysis side, first, a certain user inputs, for example, the user review 221, which is a description related to the office A used, to the user review input device. The review summarization module 301 summarizes the user review 221 and creates the indoor space state description 222.

On the other hand, the security camera 201 captures an image of a user and acquires an image of a facial expression or a gesture of the user. The image interpretation module 302 recognizes and interprets the image, and creates the current indoor space state information 232 indicating a state of an office at a time point of imaging by the security camera 201.

The space information summarization module 111 creates a recommendation tag for recommending the use of the office according to the state of the office from these indoor space state descriptions 222 and the current indoor space state information 232, and stores the recommendation tag in the dynamic database 102.

The recommendation tag includes a description that may have a mental/physical effect on the user, and is a tag related to an indoor space of the office. Examples of such a recommendation tag, for example, in the case of the office A include “# relaxing”, “# good for healing spirit”, and “# a nice rest place”, by which the comfort of the office A can be exemplified. These recommendation tags are added to a content that recommends the user to use the office. Examples of the content include a commercial that prompts a user to use an office on an internal social networking site of a company to which the user belongs or on a social network outside the company.

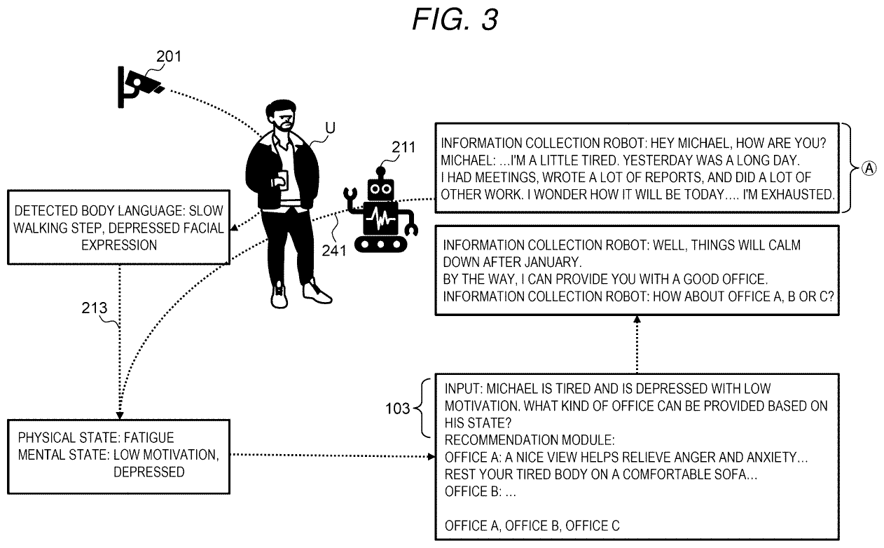

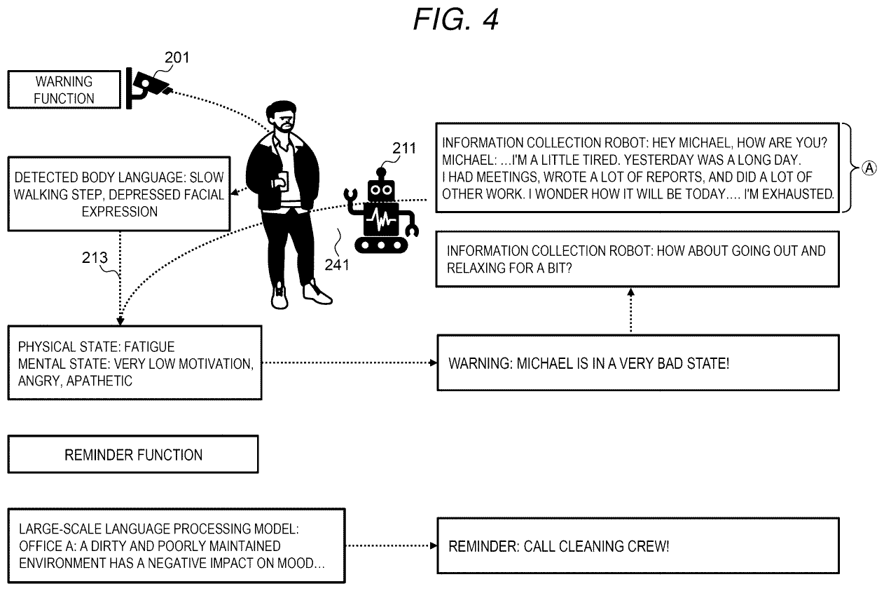

FIGS. 3 and 4 are diagrams each illustrating an example of input and output on the user analysis side in the smart building system 1000. FIG. 3 illustrates an example of basic input and output, and FIG. 4 illustrates an example of extended input and output.

First, in the example of the basic input and output illustrated in FIG. 3, for example, an image of a user U named “Michael” is captured by the security camera 201. The security camera 201 captures an image of a body language (for example, a gesture) of the user. Note that the security camera 201 may capture an image of a facial expression of the user instead of the body language of the user.

The body language interpretation module 304 interprets the body language detected by the imaging as, for example, “slow walking steps” or “depressed facial expression”, and creates the user non-oral presentation state information 213 related to the mental/physical state of the user.

On the other hand, the information collection robot 211 speaks to the user to have a conversation with the user. Examples of such a conversation include the following contents.

The information collection robot 211: “Hey Michael, how are you?”

Michael: “I'm a little tired. Yesterday was a long day. I had meetings, wrote a lot of reports, and did a lot of other work. I wonder how it will be today . . . . I'm exhausted.”

The information collection robot 211 collects the conversation with the user “Michael” (“I'm a little tired. Yesterday was a long day. I had meetings, wrote a lot of reports, and did a lot of other work. I wonder how it will be today . . . . I'm exhausted.”). The information collection robot 211 passes conversation data, which is a result of voice recognition of the conversation, to the language processing module 303. The language processing module 303 generates the user oral presentation state information 241 related to the mental/physical state of the user based on the conversation data.

The user state information summarization module 112 analyzes the physical state of the user as, for example, “fatigue” based on the user oral presentation state information 241 and the user non-oral presentation state information 213. On the other hand, the user state information summarization module 112 analyzes the mental state of the user as, for example, “low motivation” and “depressed”.

The user state information summarization module 112 generates the user state description 103 obtained by summarizing the mental/physical state of the user from these analysis results. In the present embodiment, the user state description 103 corresponds to the input of the recommendation module 101, and includes an input content such as “Michael is tired and is depressed with low motivation.”, and “What kind of office can be provided based on his state?”.

The recommendation module 101 generates the recommendation result 400 such as “Office A: A nice view helps relieve anger and anxiety . . . , Office B: . . . ” based on the user state description 103 which is the input content described above.

The recommendation module 101 generates the following recommendation result 400.

Information collection robot 211: “Well, things will calm down after January. By the way, I can provide you with a good office.”

Information collection robot 211: “How about office A, B or C?”

A specific recommendation tag including a description that the user wants to use any one of the offices is attached to the recommendation result 400 based on these conversation contents. Examples of the specific recommendation tag include “# Office A, B, C”, “# Good for mental care”, and “# Office good for changing mood”.

On the other hand, in the example of the extended input and output illustrated in FIG. 4, for example, an image of the user U named “Michael” is also captured by the security camera 201. The security camera 201 captures an image of a body language (for example, a gesture) of the user. Note that the security camera 201 may capture an image of a facial expression of the user instead of the body language of the user.

The body language interpretation module 304 interprets the body language detected by the imaging as, for example, “slow walking steps” or “depressed facial expression”, and creates the user non-oral presentation state information 213 related to the mental/physical state of the user.

On the other hand, the information collection robot 211 speaks to the user to have a conversation with the user. Examples of such a conversation include the following contents.

The information collection robot 211: “Hey Michael, how are you?”

Michael: “I'm a little tired. Yesterday was a long day. I had meetings, wrote a lot of reports, and did a lot of other work. I wonder how it will be today . . . . I'm exhausted.”

Information collection robot 211: “How about going out and relaxing for a bit?”

The information collection robot 211 collects the conversation with the user “Michael” (“I'm a little tired. Yesterday was a long day. I had meetings, wrote a lot of reports, and did a lot of other work. I wonder how it will be today . . . . I'm exhausted.”). The information collection robot 211 passes conversation data, which is a result of voice recognition of the conversation, to the language processing module 303. The language processing module 303 generates the user oral presentation state information 241 related to the mental/physical state of the user based on the conversation data.

The user state information summarization module 112 analyzes the physical state of the user as, for example, “fatigue” based on the user oral presentation state information 241 and the user non-oral presentation state information 213. On the other hand, the user state information summarization module 112 analyzes the mental state of the user as, for example, “low motivation” and “depressed”.

The user state information summarization module 112 generates the user state description 103 obtained by summarizing the mental/physical state of the user from these analysis results. In the present embodiment, the user state description 103 corresponds the to input of the recommendation module 101, and includes an input content as “Michael is tired and is depressed with low such motivation.”, and “What kind of office can be provided based on his state?”.

The recommendation module 101 indicates that the mental/physical state of the user is very bad, for example, “Warning: Michael is in such a bad state!” based on the user state description 103 which is the input content described above, analyzes the mental/physical state of the user to be bad, and generates, based on the analysis result, the recommendation result 400 to which a specific recommendation tag that suggests, for example, “How about going out and relaxing for a bit?”, which is a recommendation content that can resolve the bad state, has been added.

In the recommendation result 400, examples of a specific recommendation tag include “# If you go out and relax, office A is clean and comfortable.”, in addition to the description content described above.

The smart building system 1000 according to the present embodiment includes: an input device, the security camera 201, the information collection robot 211, or the like as an example of an indoor space monitoring unit that monitors a state of an indoor space; the review summarization module 301 and the image interpretation module 302 as an example of an indoor space state analysis unit that analyzes the state of the indoor space and acquires the indoor space state description 222 and the current indoor space state information 232 as an example of indoor space state information as an analysis result; the security camera 201 and the information collection robot 211 as an example of a user monitoring unit that monitors a mental/physical state of a user in the indoor space; the language processing module 303 and the body language interpretation module 304 as an example of a user state analysis unit that analyzes the mental/physical state of the user and acquires the user oral presentation state information 241 and the user non-oral presentation state information 213 as an example of user state information as an analysis result; and a space information summarization module 111 as an example of a recommendation tag creation unit that creates, based on the indoor space state description 222 and the current indoor space state information 232 as an example of indoor space state information and the user oral presentation state information 241 and the user non-oral presentation state information 213 as an example of user state information, a specific recommendation tag related to the indoor space that may affect a behavior of the user.

An indoor space provision method of the smart building system 1000 according to the present embodiment includes: an indoor space monitoring step of monitoring a state of an indoor space by an indoor space monitoring unit; an indoor space state analysis step of analyzing the state of the indoor space and acquiring indoor space state information as an analysis result by an indoor space state analysis unit; a user monitoring step of monitoring a mental/physical state of a user in the indoor space by a user monitoring unit; a user state analysis step of analyzing the mental/physical state of the user and acquiring user state information as an analysis result by a user state analysis unit; and a recommendation tag creation step of creating, based on the indoor space state information and the user state information, a specific recommendation tag related to the indoor space that may affect a behavior of the user by a recommendation tag creation unit.

With such a configuration, when the user comes into contact with the specific recommendation tag, it is possible to recommend an indoor space suitable for the mental/physical state of the user, which can change the mood of the user by the mental/physical effect of the description content of the specific recommendation tag.

In the present embodiment, the indoor space state analysis unit includes the review summarization module 301 that summarizes a user review related to the indoor space and acquires an indoor space state description as indoor space state information, which is a description described by the user, the image interpretation module 302 that acquires the current indoor space state information 232 as indoor space state information related to the indoor space monitored by the indoor space monitoring unit, and a space information summarization module 111 that creates, based on the indoor space state description 222 and the current indoor space state information 232, the specific recommendation tag related to the indoor space. In this way, the indoor space suitable for the mental/physical state of the user can be recommended to the user who has come into contact with the specific recommendation tag created based on the indoor space state description 222 and the current indoor space state information 232.

In the present embodiment, the user state analysis unit includes the language processing module 303 that executes language processing of a conversation uttered by the user and creates the user oral presentation state information 241 which is information related to a state of the user according to an oral presentation content of the user, and the body language interpretation module 304 that interprets a body language of the user and creates user non-oral presentation state information 213 which is information related to a state of the user according to a non-oral presentation content of the user. In this way, it is possible to recommend an indoor space suitable for the mental/physical state of the user to the user who has come into contact with the specific recommendation tag created based on the user oral presentation state information 241 and the user non-oral presentation state information 213.

In the present embodiment, the user state analysis unit includes the user state information summarization module 112 that creates the user state description 103 related to the mental/physical state of the user based on the user oral presentation state information 241 and the user non-oral presentation state information 213, and the smart building system 1000 includes the recommendation module 101 as an example of a recommendation unit that provides the user with a specific recommendation tag created by the space information summarization module 111 as an example of a recommendation tag creation unit when the specific recommendation tag matches a previous recommendation tag created in the past in consideration of the user state description 103. In this way, it is possible to recommend an indoor space suitable for the mental/physical state of the user to the user who has come into contact with the more effective specific recommendation tag.

In the present embodiment, the language processing module 303 and the body language interpretation module 304 as an example of the user state analysis unit estimate the mental/physical state of the user based on a result of processing the content of a conversation of the user as a monitoring result by a user state monitoring unit using natural language processing. The user state analysis unit may use artificial intelligence instead of the natural language processing. In this way, it is possible to recommend an indoor space suitable for the mental/physical state of the user to the user who has come into contact with the specific effective recommendation tag from the content of the conversation of the user.

The invention is not limited to the embodiment described above, and includes various modifications and equivalent configurations within the scope of the appended claims. For example, the above-described embodiment has been described in detail to facilitate understanding of the invention, and the invention is not limited to those including all the above-described configurations. At least one of the elements described as being connected in parallel in the present embodiment may be connected in series to another element.

### INDUSTRIAL APPLICABILITY

The invention can be applied to, for example, an indoor space provision system related to a technique for providing an indoor space comfortable for a user.

What is claimed is:

1. An indoor space provision system comprising:
an indoor space monitoring unit configured to monitor a state of an indoor space;
an indoor space state analysis unit configured to analyze the state of the indoor space and acquire indoor space state information as an analysis result;
a user monitoring unit configured to monitor a mental/physical state of a user in the indoor space;
a user state analysis unit configured to analyze the mental/physical state of the user and acquire user state information as an analysis result; and
a recommendation tag creation unit configured to create, based on the indoor space state information and the user state information, a specific recommendation tag related to an indoor space that affects a behavior of the user.

2. The indoor space provision system according to claim 1, wherein
the indoor space state analysis unit includes
a review summarization module configured to summarize a review related to the indoor space and acquire an indoor space state description as the indoor space state information, the review being a description described by the user,
an image interpretation module configured to acquire current indoor space state information as the indoor space state information related to the indoor space monitored by the indoor space monitoring unit, and
a space information summarization module configured to create the specific recommendation tag related to the indoor space based on the indoor space state description and the current indoor space state information.

3. The indoor space provision system according to claim 1, wherein
the user state analysis unit includes
a language processing module configured to execute language processing of a conversation uttered by the user and create user oral presentation state information which is information related to a state of the user according to an oral presentation content of the user, and
a body language interpretation module configured to interpret body language of the user and create user non-oral presentation state information which is information related to a state of the user according to a non-oral presentation content of the user.

4. The indoor space provision system according to claim 3, wherein
the user state analysis unit includes a user state information summarization module configured to create a user state description related to the mental/physical state of the user based on the user oral presentation state information and the user non-oral presentation state information, and
the indoor space provision system includes a recommendation unit configured to provide the user with the specific recommendation tag created by the recommendation tag creation unit when the specific recommendation tag matches a previous recommendation tag created in the past in consideration of the user state description.

5. The indoor space provision system according to claim 1, wherein
the user state analysis unit estimates the mental/physical state of the user based on a result of processing a content of the conversation of the user as a monitoring result by the user state monitoring unit using natural language processing.

6. An indoor space provision method comprising:
an indoor space monitoring step of monitoring a state of an indoor space by an indoor space monitoring unit;
an indoor space state analysis step of analyzing the state of the indoor space and acquiring indoor space state information as an analysis result by an indoor space state analysis unit;
a user monitoring step of monitoring a mental/physical state of a user in the indoor space by a user monitoring unit;
a user state analysis step of analyzing the mental/physical state of the user and acquiring user state information as an analysis result by a user state analysis unit; and
a recommendation tag creation step of creating, based on the indoor space state information and the user state information, a recommendation tag related to the indoor space that affects a behavior of the user by a recommendation tag creation unit.

7. The indoor space provision method according to claim 6, wherein
the indoor space state analysis step includes
a review summarization step of summarizing a review related to the indoor space and acquiring an indoor space state description as the indoor space state information by a review summarization module, the review being a description described by the user,
an image interpretation step of acquiring, by an image interpretation module, current indoor space information as the indoor space state information related to the indoor space monitored by the indoor space monitoring unit, and
a space information summarization step of creating, by a space information summarization module, the specific recommendation tag related to the indoor space based on the indoor space state description and the current indoor space state information.

8. The indoor space provision method according to claim 6, wherein
the user state analysis step includes
a language processing step of executing language processing of a conversation uttered by the user and creating user oral presentation state information which is information related to a state of the user according to an oral presentation content of the user by a language processing module, and
a body language interpretation step of interpreting body language of the user and creating user non-oral presentation state information which is information related to a state of the user according to a non-oral presentation content of the user by a body language interpretation module.

9. The indoor space provision method according to claim 8, wherein
the user state analysis step includes a user state information summarization step of creating, by the user state information summarization module, a user state description related to the mental/physical state of the user based on the user oral presentation state information and the user non-oral presentation state information, and
a recommendation unit provides the user with the specific recommendation tag created by the recommendation tag creation unit when the specific recommendation tag matches a previous recommendation tag created in the past in consideration of the user state description.

10. The indoor space provision method according to claim 6, wherein
in the user state analysis step, the user state analysis unit estimates the mental/physical state of the user based on a result of processing a content of the conversation of the user as a monitoring result by the user state monitoring unit using natural language processing.
